(19) United States
(12) Patent Application Publication
(10) Pub. No.: US 2024/0142433 A1
(43) Pub. Date: May 2, 2024

(54) METHOD FOR DETECTING AROMATIC HYDROCARBONS AND/OR DIAMONDOIDS USING FOURIER TRANSFORM ION CYCLOTRONIC RESONANCE MASS SPECTROMETRY COUPLED WITH THE ATMOSPHERIC PRESSURE PHOTOIONIZATION SOURCE

(71) Applicants: PETRÓLEO BRASILEIRO S.A. - PETROBRAS, Rio de Janeiro (BR); UNIVERSIDADE FEDERAL DE GOIÁS, Goiânia (BR)

(72) Inventors: ALEXANDRE DE ANDRADE FERREIRA, Rio de Janeiro (BR); YGOR DOS SANTOS ROCHA, Rio de Janeiro (BR); CARLOS ALBERTO CARBONEZI, Rio de Janeiro (BR); RODRIGO CABRAL DA SILVA, Rio de Janeiro (BR); JOELMA PIMENTEL LOPES, Rio de Janeiro (BR); BONIEK GONTIJO VAZ, Goiânia (BR); TAYNARA COVAS RODRIGUES, Goiânia (BR); DANIELLE MITZE MULLER FRANCO, Goiânia (BR); MARIO DUNCAN RANGEL, Goiânia (BR); ROSANA CARDOSO LOPES PEREIRA, Goiânia (BR)

(21) Appl. No.: 18/478,281

(22) Filed: Sep. 29, 2023

(30) Foreign Application Priority Data
Sep. 30, 2022 (BR) 10 2022 019814 4

(51) Int. Cl. G01N 33/28; G01N 1/38; H01J 49/38

(52) U.S. Cl. CPC G01N 33/2835; G01N 1/38; G01N 33/2823; H01J 49/38

(57) ABSTRACT

The present invention relates to the field of organic geochemistry wherein a method for accessing high molecular mass aromatic hydrocarbons and diamondoids was developed from comprehensive characterization carried out by high resolution spectrometry coupled with the atmospheric pressure photoionization source (APPI FT-ICR MS). Based on the compositional profile of diamondoids and aromatic hydrocarbons, it is possible to quickly and robustly classify oils in relation to their origin and thermal evolution. It is verified that the compositional detail provided by the APPI(+)-FT-ICR MS analysis allowed the development of new molecular indicators, accessed without the need for any preliminary separation technique, in order to become a powerful tool for prospecting the use of oils exploited for specific purposes.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the benefit of Brazilian Application No. BR 10 2022 019814 4, filed 30 Sep. 2022, the entire contents of which are hereby incorporated by reference.

### FIELD OF THE INVENTION

The present invention relates to the field of organic geochemistry wherein a method for accessing high molecular mass aromatic hydrocarbons and diamondoids was developed from comprehensive characterization carried out by high resolution spectrometry coupled with the atmospheric pressure photoionization source (APPI FT-ICR MS). Based on the compositional profile of diamondoids and aromatic hydrocarbons, it is possible to quickly and robustly classify oils in relation to their origin and thermal evolution. It is verified that the compositional detail provided by the APPI(+)-FT-ICR MS analysis allowed the development of new molecular proxies, accessed without the need for any preliminary separation technique, in order to become a powerful tool for prospecting the use of oils exploited for specific purposes.

### BACKGROUND OF THE INVENTION

In the last 20 years, mass spectrometry (MS) has undergone great development. One of the highlights was in relation to analyzers, with the advent of mass spectrometry of very high resolution and accuracy (FT-MS, Fourier transform mass spectrometry), initially represented by Fourier transform ion cyclotron resonance mass spectrometry (FT-ICR MS) (Marshall, Hendrickson and Jackson, 1998). These new techniques and equipment make it possible to characterize polar compounds extremely quickly and efficiently. Oils of different origins, biodegradation levels (Vaz, et al., 2013) and thermal maturation (Rocha, et al., 2018) have presented very distinct and characteristic profiles. As FT-ICR MS is a comprehensive characterization technique, its results can be used both as an aid to exploration and production, refining and distribution activities (Hughey, et al., 2001; Smith, et al., 2007; Dalmaschio, et al., 2014).

The coupling of FT-ICR MS with atmospheric pressure ionization techniques, such as electrospray ionization (ESI), atmospheric pressure chemical ionization (APCI) and atmospheric pressure photoionization (APPI), made it possible to characterize polar and even non-polar compounds (aromatic hydrocarbons) extremely quickly and efficiently. The success of FT-ICR MS for the study of oil and oil derivatives is due not only to advances in instrumentation (analyzer and ionization techniques) but also to data processing methods. In relation to these, it is necessary to use specific software for processing the spectra, such as Composer. Such software, in addition to assigning molecular formulas for each mass/charge relation (m/z), has a set of graphical tools that allow all compositional information to be displayed in a clearer and more visual way. Graphical tools such as: class diagrams and DBE vs carbon number graphs are routinely the most used (Hsu, Qian and Chen, 1992; Kim, Kramer and Hatcher, 2003). Ternary diagrams, DBE distribution graphs, total carbon, among others can be obtained by using specific software, such as Thanus. Such diagrams and graphs constitute essential tools for interpreting and analyzing data trends.

Despite all the development experienced by petroleomics in recent years, methodological developments are necessary to overcome the challenges that still exist, mainly in the establishment of new parameters for the geochemical evaluation of oils. In recent years, there have been few petroleomics studies applied to organic geochemistry. The few studies focus on the global evaluation of the composition as a function of a geochemical process, that is, biodegradation (Vaz, et al., 2013), thermal evolution (Oldenburg, et al., 2014), lithofacies (Silva, et al., 2020), migration (Poets, et al., 2019) or origin (Rocha, et al., 2018). The need to establish new parameters and in-depth evaluation of results for proper use as a tool for geochemical classifications is evident (Asemani and Rabbani, 2020).

### STATE OF THE ART

When analyzing the state of the art, documents were found that disclose procedures, methods and techniques normally used for the compositional analysis of oil. Although the documents found do not disclose all the main features of the present invention, it is clear that the matter in question arouses special interest in the scientific community, since the use of Fourier transform ion cyclotron resonance mass spectrometry (FT-ICR MS) coupled with the atmospheric pressure photoionization (APPI) source has shown considerable potential for the detection, not only of aromatic hydrocarbons, but also for the geochemical/compositional characterization of oil, as noted below.

In US patent document 8,932,863 B2, methods for evaluating a fuel by identifying trace components therein are disclosed. According to that document, a method of evaluating a fuel includes providing a test sample of the fuel. In addition, the method includes analyzing the test sample and identifying a trace compound in the test sample. The method also determines whether the fuel is of biological origin based on the trace compound identified. Said analysis includes ionizing the test sample by an atmospheric pressure photoionization (APPI) source, followed by verification with Fourier transform ion cyclotron resonance mass spectrometry (FT-ICR MS).

Despite the similarity due to the use of the APPI (+) FT-ICR MS source, it is clear that the method is limited to discriminating the raw material for fuel production, being applied with the purpose of differentiating fossil fuels from those of biological origin, such as biodiesel and related materials. Therefore, there is no indication in U.S. Pat. No. 8,932,863 B2 that discloses, not even by logical inference, the method for detecting aromatic hydrocarbons and/or diamondoids with the purpose of detailing the composition of hydrocarbons and using the same for geochemical classification of oils.

Document U.S. Pat. No. 10,725,013 B2 refers to a method for the evaluation of oil samples and their fractions by Fourier transform ion cyclotron resonance mass spectrometry (FT-ICR MS), using, among other techniques, the atmospheric pressure photoionization (APPI) as an ionization source.

In document U.S. Pat. No. 10,725,013 B2, again, there is as similarity of using the APPI (+) FT-ICR MS source, but with another purpose. A detailed characterization of the molecular composition of oils and heavy oil fractions is observed by APPI(+)-FT-ICR MS. However, the main object of U.S. Pat. No. 10,725,013 B2 refers to the method that consists of using the molecular composition obtained by APPI(+)-FT-ICR MS to determine oil properties such as: cetane number, pour point, cloud point, aniline point and octane number in the gasoline fraction. These correlations provided information on oil to gas properties without fractionation/distillation, bringing valuable information for production, refining and oil quality markers. Several equations have been proposed for this purpose. However, at no time were aromatic hydrocarbons used to evaluate the thermal evolution of oils, and there was no reference to the geochemical/compositional characterization of oil, not even to the markers proposed by the present invention.

Document WO2020257277A1 refers to a system and method for characterizing oil and its derivatives by combining total sulfur determination and Fourier transform ion cyclotron resonance mass spectrometry (FT-ICR MS), which may be equipped with atmospheric pressure photoionization (APPI). In addition, such a document states that it is the first time that aromatic compounds have been determined by APPI FT-ICR MS.

In document WO2020257277A1, there is as similarity in the use of the APPI (+) FT-ICR MS source, as well as the evaluation of the aromatic hydrocarbon fraction of the oils. The difference in relation to the present invention lies in the application of the set of data generated, that is, the compounds ionized by this technique. In reference WO2020257277A1, a method for determining the main characteristics of hydrocarbon (saturated and aromatic) and sulfur fractions is proposed. The method, in short, consisted of carrying out a “group-type” analysis using APPI (+) FT-ICR MS data. As a result, the mass fraction of sulfur and aromatic compounds was obtained, useful information to support refining actions. At no time were aromatic hydrocarbons used to evaluate the thermal evolution of oils, a focus of geochemical application, nor the markers proposed in the present invention.

Document CN107643357B refers to a method for the analysis of sterane-based compounds in a geological sample of oil. According to this method, organic materials in a rock core sample are extracted with an organic solvent and the supernatant is analyzed using Fourier transform ion cyclotron resonance mass spectrometry coupled with an atmospheric pressure photoionization source (APPI FT-ICR MS).

Despite the similarity with the use of the APPI (+) FT-ICR MS source and the analysis of steranes, the present invention differs from document CN107643357B by the application of the set of data generated, that is, the compounds ionized by this technique, which in this patent proposed a method for extracting steranes from rock using organic solvent and subsequent analysis directly by APPI(+) FT-ICR MS. Steranes are polycyclic hydrocarbons with the general formula CnH2n-6 (n>3). Although steranes are markers for studies in organic geochemistry, the strategy employed in the method of the present invention was never used.

Document JP2021162365 A presents an analytical method for heavy oil fractions using Fourier transform ion cyclotron resonance mass spectrometry coupled with an atmospheric pressure photoionization source (APPI FT-ICR MS).

Despite the similarity with the use of the APPI (+) FT-ICR MS source, the present invention differs from document JP2021162365 A by the application of the set of data generated, that is, the compounds ionized by this technique, which in this patent proposed a method for analyzing heavy fractions of oil by examining the sulfur content present. JP2021162365 A does not present similarity with the methodological strategy used in the present invention for the use of hydrocarbons in organic geochemistry studies.

The paper by BAE et al. (2010), Identification of about 30 000 Chemical Components in Shale Oils by Electrospray Ionization (ESI) and Atmospheric Pressure Photoionization (APPI) Coupled with 15T Fourier Transform Ion Cyclotron Resonance Mass Spectrometry (FT-ICR MS) and a Comparison to Conventional Oil. Energy Fuels 2010, 24, 2563-2569: DOI:10.1021/ef100060b, refers to the identification of chemical components, including aromatic hydrocarbons, in shale oil using electrospray ionization (ESI) and atmospheric pressure photoionization (APPI) coupled to Fourier transform ion cyclotron resonance mass spectrometry (FT-ICR MS).

Although there is similarity with the use of the APPI (+) FT-ICR MS source, the present invention differs from the document by BAE et al. by the application of the generated data set, that is, of the compounds ionized by this technique, which in this paper, general characterizations of three oils were carried out by relative abundance, length of the alkyl chain and class unsaturations. This paper at no time discloses the use of aromatic hydrocarbons for the purpose of the present invention, which involves application in organic geochemistry studies.

The paper by PEREIRA, T. M. C. (2013), Aplicaøões da espectrometria de massas de ressonância ciclotrônica de íons por transformada de Fourier (FT-ICR MS) em petroleomica (Applications of Fourier transform ion cyclotron resonance mass spectrometry (FT-ICR MS) in petroleomics) (Master's thesis in Chemistry—UFES), refers to spectrometry applications of Fourier transform ion cyclotron resonance mass in Petroleomics using various ionization sources, among them, photoionization at atmospheric pressure for the acquisition of data related to the characterization of asphaltenes and oil samples.

In the paper by PEREIRA, T. M. C. (2013), there is as a similarity the use of the APPI source (+) FT-ICR MS. However, the present invention differs from the aforementioned paper by the application of the set of data generated, that is, the compounds ionized by this technique, which in this master's thesis was the use of the method for analyzing asphaltenes and acidic oil fractions. The aforementioned master's thesis at no point discloses the use of aromatic hydrocarbons for application in organic geochemistry studies.

The paper by PURCELL et al. (2009) Stepwise Structural Characterization of Asphaltenes during Deep Hydroconversion Processes Determined by Atmospheric Pressure Photoionization (APPI) Fourier Transform Ion Cyclotron Resonance (FT-ICR) Mass Spectrometry. Energy Fuels 2010, 24, 2257-2265: DOI:10.1021/ef900897a, refers to the structural characterization of asphaltenes during deep hydroconversion process determined by Fourier transform ion cyclotron resonance mass spectrometry coupled with atmospheric pressure photoionization source (APPI FT-ICR MS). According to this document, such techniques provide detailed molecular characterization for, among others, the hydrocarbon asphaltene.

In the paper by PURCELL et al. (2009), despite using the APPI (+) FT-ICR MS source to monitor a conversion in asphaltenes process and detailing the composition of the fractions analyzed in terms of carbon number, DBE and classes, there is no inference regarding the use of any reason for classifying the thermal evolution of oils, a focus of geochemical application, nor of the markers proposed in the present invention.

The paper by ROGERS & MCKENNA (2011), Petroleum Analysis, Anal. Chem. 2011, 83, 4665-4687 (2011), dx.doi.org/10.1021/ac201080e is a literature review of the main techniques used in oil analysis. Among the mentioned techniques, the use of Fourier transform ion cyclotron resonance mass spectrometry coupled with an atmospheric pressure photoionization source for detailed compositional analysis of hydrocarbons and crude oil is mentioned.

This review mentions the use of the APPI (+) FT-ICR MS source for evaluating the composition of oils and distillation products, showing an increase in aromatization and carbon number of the hydrocarbons with increasing distillation cutoff proving Boduszynski's prediction was right. However, at no time was the use of this technique mentioned for geochemical evaluations using hydrocarbons.

From the analysis of the state of the art, it was possible to observe that, despite the similarity in the use of the APPI (+) FT-ICR MS ionization source for the analysis of oil and by-products, in all documents found it was used the same ionization source but with focus on different applications and in different fields, without impairing the inventive nature of the present invention, nor even bringing precepts that, added to technical knowledge and combined with other available data, could directly lead to the present invention. No document shows the analysis of aromatic hydrocarbon compounds applied to geochemical studies, nor even the reference to the compounds used in the present invention for the geochemical classification of oils in relation to thermal evolution.

It is reiterated that the present invention has as its main focus the analysis at the elementary level of hydrocarbon compounds in order to provide a direct method, without the need for prior fractionation, or compound concentration steps, as is traditionally carried out. The method presented herein has several adjustments to the spectra acquisition parameters in the mass spectrometer in order to provide direct access to compounds such as diamondoids (adamantane and diamantane).

Furthermore, an extremely important step is data processing. The strategy used guaranteed access to compounds of great value for geochemical applications. It should be emphasized here that after extensive scrutiny in the literature, there is no report on the analysis of these compounds by direct ionization from oil. The method proposed herein, consisting of several steps, includes oil data acquisition parameters, data processing and establishment of ratios, “proxies”, for geochemical classification of oils in relation to the thermal evolution.

This new method for evaluating aromatic hydrocarbons, in addition to diamondoids, presents the evaluation of other markers such as phenanthrene, naphthalene, and mono- and triaromatic steranes for direct application in organic geochemistry to determine the thermal evolution of oils and condensates.

Therefore, as in the cited references that rely on the APPI method coupled to Fourier transform ion cyclotron resonance mass spectrometry, but employing different strategies for specific objectives, the present invention uses the same technique, although with a purpose and modus operandi different than that previously reported. In this way, the invention preserves the new and inventive character in face of the aforementioned state of the art.

### BRIEF DESCRIPTION OF THE INVENTION

The present invention relates to a method for the direct detection of diamondoids and a plurality of aromatic hydrocarbons by the APPI (+) FT-ICR MS technique, previously accessed by methods that require several laborious steps such as fractionation, extraction, in addition to chromatographic analysis of fractions and extracts. Furthermore, the invention, based on the compositional profile of diamondoids and aromatics, allows the classification of oils in relation to origin and thermal evolution with high speed and robustness. Therefore, the invention contributes to greater economy, since the proposed method employs the direct analysis of crude oil, without prior steps of sample preparation. Finally, the great compositional detail provided by APPI(+)-FT-ICR MS analysis allowed the development of new molecular parameters for geochemical characterization of oils in relation to origin and thermal evolution with great reliability. The new molecular parameters discussed in the present invention, accessed without the need for any preliminary separation technique, can become a powerful tool for prospecting the use of explored oils for specific purposes.

The comprehensive molecular characterization of oils by APPI(+) FT-ICR MS allows obtaining indices for evaluating the thermal evolution of fluids, oil, from oil reservoirs. The thermal evolution is an important geochemical parameter used to describe the history of accumulation and, mainly, to support basin modeling that allows the exploration potential to be leveraged while minimizing risks.

The two indices, proxies, fruits of this invention, will allow qualitatively access to the thermal evolution of oils of different origins. Traditionally, the thermal evolution is established using numerous molecular markers, each accessed by a specific method, which makes the analysis complex. The indices proposed herein proved to be robust and are produced by a direct APPI (+) FT-ICR MS analysis of the crude oil.

### BRIEF DESCRIPTION OF THE FIGURES

In order to complement the present description and obtain a better understanding of the features of the present invention, and in accordance with a preferred embodiment thereof, in annex, a set of figures is presented, where in an exemplified, although not limiting, manner, its preferred embodiment is represented.

FIG. 1a shows mass spectra obtained by APPI(+) FT-ICR MS of a representative oil sample.

FIG. 1b shows mass spectra obtained by class diagram of a representative oil sample.

FIG. 2 represents the distribution of carbon number of the HC class by the relative abundance of DBE 7 and 10, related to naphthalene and phenanthrene, respectively, in samples from the Santos Basin.

FIG. 3 represents the scatter plot of the ratio of phenanthrene divided by the sum of phenanthrene with methylphenantrene (C14/C14+C15, DBE 10) with the sum of atoms from C13 to C₁₆ (propyl, butyl and pentylnaphthalene) of DBE 7 divided by the sum of the carbon number relative to DBE 7, in samples from the Santos basin.

FIG. 4 represents the schematic of the biosynthetic path for the transformation of tricyclic diterpenoids into phenantrene, via simonellite and retene. The structures highlighted in red were those used to monitor the relative abundances of these structures with the advancement of the thermal evolution in samples from the Santos Basin.

FIG. 5 presents the structures of monoaromatic and triaromatic steroids with carbon numbers C17 and C27.

FIG. 6 represents the scatter plot using the ratios of triaromatic steroids divided by the sum of monoaromatic and triaromatic steroids (TA/TA+TM), using C17 and C27 for samples from the Santos Basin.

FIG. 7 presents the CID MS-MS experiments of a) ethyladamantane (C12H20), b) diamantane (C14H20).

FIG. 8 represents the graph of the carbon number distribution by the relative abundance of hydrocarbons of DBE 3 (adamantane), 5 (diamantane) and 7 (triamantane) for the samples from the Santos Basin.

FIG. 9 illustrates the scatter plot of the ratio of Ts/Ts+Tm for ethyladamantane (a), diamantane (b) and triamantane (c), for samples from the Santos Basin.

FIG. 10 represents the scatter plot of the sum of C13-C16 (naphthalene, DBE 7) by the sum of the total carbon number of DBE 7 of the HC class, from the Santos Basin samples.

FIG. 11 represents the carbon number distribution graph for DBE 5 (diamantane), DBE 7 (naphthalene) and DBE 10 (phenanthrene) for Type II-S kerogen hydropyrolysis samples.

FIG. 12a illustrates the scatter plot of the ratio of the sum of C13 to C₁₆ (naphthalene, DBE 7) divided by the sum of the total carbon number of DBE 7 by the ratio of diamantane divided by the sum of diamantane and methyldiamantane.

FIG. 12b illustrates the scatter plot of the ratio of the sum of C14 to C17 (phenanthrene, DBE 10) by the sum of the total carbon number of DBE 10 by the ratio of the sum of C13 to C₁₆ (naphthalene, DBE 7) divided by the sum of the total carbon number of DBE 7, both from Hydropyrolysis samples.

FIG. 13 presents the scatter plot using the ratios of triaromatic steroids divided by the sum of monoaromatic and triaromatic steroids (TA/TA+TM), using the C17 and C27 atoms for the Hydropyrolysis samples.

FIG. 14 presents the components of the method that integrates the invention of two new proxies for evaluating the thermal evolution of oils.

FIG. 15 presents the general scheme of preparing oil samples for APPI(+) FT-ICR MS analyses.

### DETAILED DESCRIPTION OF THE INVENTION

The prospecting of markers for geochemical characterization of oil using advanced mass spectrometry techniques, especially Fourier transform ion cyclotron resonance mass spectrometry, FT-ICR MS, is a current challenge in organic geochemistry of the oil. What is currently routine marker analysis is the application of conventional mass spectrometry to investigate specific molecules that provide information, for example, about the origin and degree of thermal evolution of condensed oils.

Over the last few decades, several biomarkers from the classes of hopanes, steranes and diamondoids have been used with the purpose of geochemically characterizing this type of sample, assisting in the study of oil systems. However, there is still no set of molecular parameters considered absolute and infallible in the geochemical characterization of oil, especially those associated with the thermal maturation thereof.

FT-ICR MS, despite not discriminating specific molecules, is being proposed as an alternative and robust tool for investigating the level of thermal evolution of oils and condensates, through the analysis of diamondoids and aromatic biomarkers, and using a source with APPI ionization (+), which directly ionizes nonpolar ions such as hydrocarbons.

Experimentally, there is no report in the literature (scientific paper or patent) of the identification of diamondoids and aromatic compounds by atmospheric pressure ionization methods coupled with FT-ICR MS. This opens the door for the establishment of new molecular parameters for the geochemical characterization of oils, even for the classification of oils in terms of paleodepositional aspects.

The compositional characterization of oils using petroleomics strategies allows access to thousands of potential markers. The ionization method by photoionization at atmospheric pressure is a method that can access low, medium and higher polarity molecules in oil. It is a method that presents good reproducibility and repeatability and is therefore credible to be used as a standard method for establishing new protocols for the geochemical characterization of oil.

When coupled to FT-ICR MS, it makes it possible to access thousands of chemical constituents, a number greater than any other analytical method. Based on the disclosure, the present invention therefore addresses to the development of a method for direct characterization, without fractionation and chromatographic elution, of aromatic hydrocarbons and diamondoids using the atmospheric pressure photoionization (APPI) ionization technique combined with Fourier transform resonance mass spectrometry ion cyclotronic (FT-ICR MS) and its application in geochemical characterizations of oils.

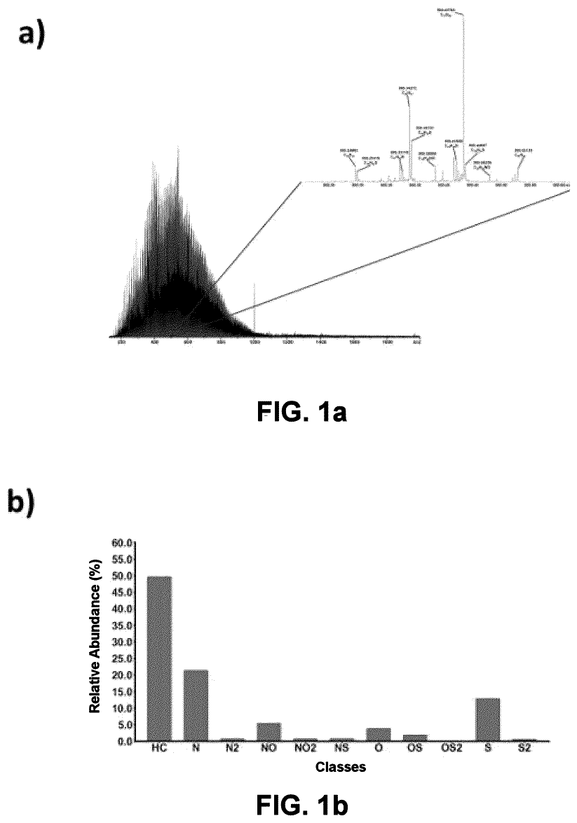

A wide set of oils was analyzed by APPI (+) FT-ICR MS. The spectra were acquired with a resolving power R=800,000.00. FIG. 1a illustrates a typical APPI(+) FT-ICR MS spectrum of a crude oil. FT-ICR MS analyses achieve such a resolving power that allows the assignment of molecular formulas unequivocally. The preliminary evaluation of the composition of NSO obtained by APPI (+) is illustrated in FIG. 1b. Note that the HC class, hydrocarbons, is the majority class, followed by the N and S classes. However, it is not possible to identify isomers of naphthalene, phenanthrene, which are normally used in thermal evolution studies of oils and oil extracts.

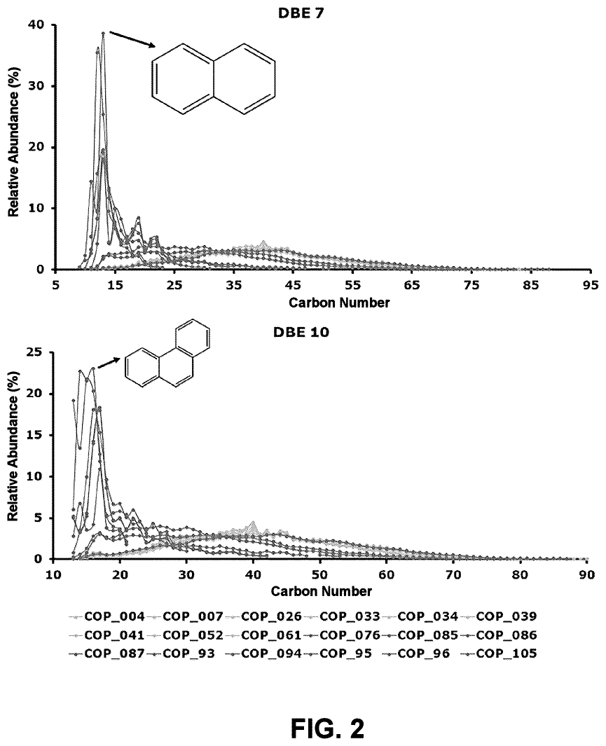

In FIG. 2, there can be observed the distribution of carbon number by relative abundance that shows an emergence of structures with a lower number of carbon atoms in more thermally evolved samples. In naphthalene, molecular formula C10H8, corresponding to DBE 7, the increase is evident in structures from C13 to C16. In phenantrene, C14H10, DBE 10, there can be observed the same more intense relative abundance in carbons from C14 to C18. The distribution of carbon number by relative abundance discloses an emergence of structures with fewer carbon atoms in more thermally evolved samples. In naphthalene, molecular formula C10H8, corresponding to DBE 7, the increase is evident in structures from C13 to C16. In phenantrene, C14H10, DBE 10, there can be observed the same more intense relative abundance in carbons from C14 to C18.

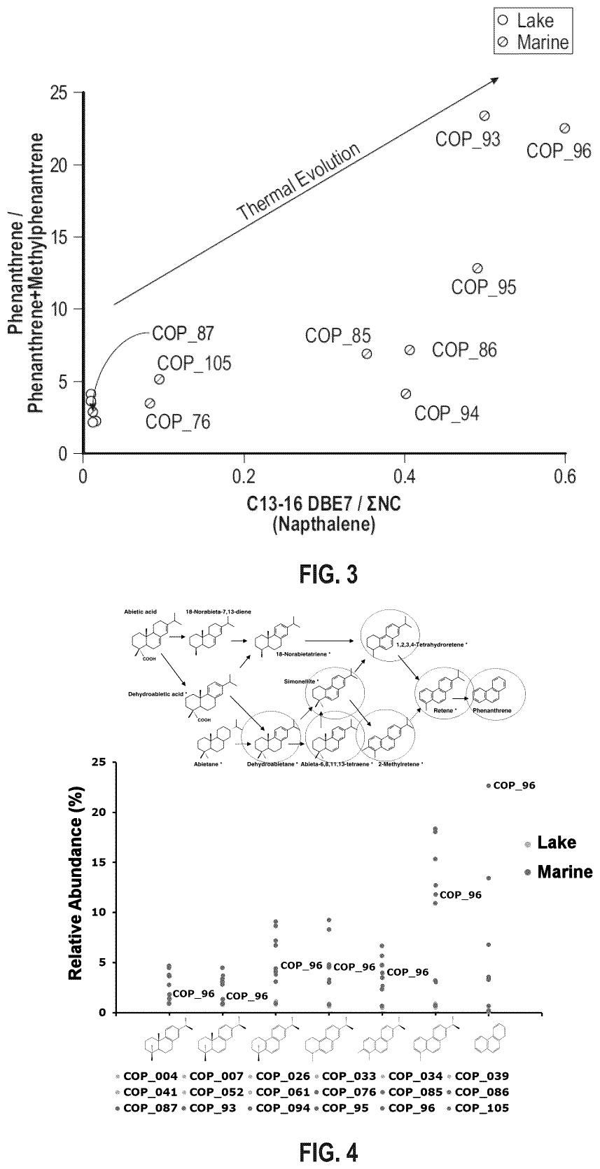

In FIG. 3, there is presented the graph of the ratio of phenanthrene divided by the sum of phenanthrene with methylphenanthrene, C14/C14+C15, DBE 10 (normalized by DBE 10) by the ratio of the sum of carbon atoms from C13 to C16 (propyl, butyl or pentinaphthalene), DBE 7 (normalized by DBE 7), divided by the sum of the carbon number relative to DBE 7. With the ratios using naphthalene and phenanthrene there is achieved a thermal evolution trend very similar to that of mono and triaromatic steroids presented previously. However, samples COP 93 and 96 using naphthalene and phenanthrene markers showed the highest thermal evolution compared to samples of marine origin, which was not observed using mono and triaromatic steroids, as they were not ionized.

In thermal evolution studies, the reasons involving phenanthrene and methylphenantrene are already established in the literature. Through the abietic acid biosynthetic path, tetracyclic diterpenes are transformed into phenanthrenes through retene and simonellite. This biosynthetic path can be followed using the APPI ionization source; however, this source does not effectively ionize ions of the O2 class, the precursor of this path. In this way, the relative abundances of structures from dehydroabiethene to phenanthrene were evaluated, highlighted in red in FIG. 4 (top). In FIG. 4, there can be observed a relative increase in phenanthrene in more thermally evolved samples. In sample COP_96, the most evolved sample of this group of samples, there can be observed that there is a decrease in less aromatic structures, dehydroabiethene, with the increase in thermal evolution and with this a relative increase in the more aromatic structures, phenanthrene. This finding demonstrates the potential of this analytical technique, corroborating the study carried out by Haberer et al., 2006, in which the GC-MS technique was used.

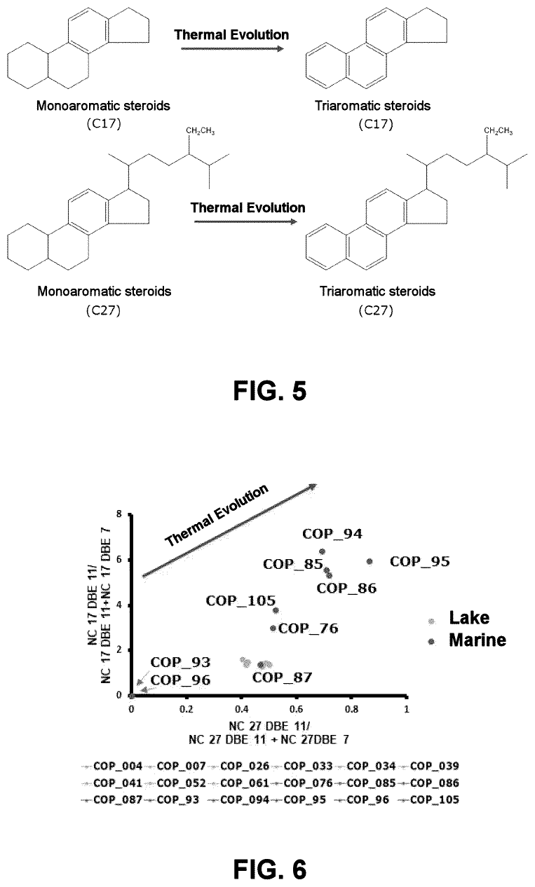

FIG. 5 illustrates the structures of monoaromatic and triaromatic steroids, showing both the structure with 17 and 27 carbon atoms and highlights the aromatization that occurs in the naphthenic rings from monoaromatic s to triaromatics with the thermal evolution.

FIG. 6 illustrates the ratio of triaromatic steroids divided by the sum of monoaromatic and triaromatic steroids (TA/TA+TM) presenting a good correlation for evaluating the thermal evolution of samples when considering both compounds with carbon number C17 and the C27. It is noted for samples from marine origin that COP 87 presents the lowest thermal evolution and COP 94 and 95 the highest. Samples COP 93 and 96 did not present ions corresponding to these structures, perhaps due to the high thermal cracking thereof, whereas the samples from lake origin showed very similar thermal evolution.

Diamondoids

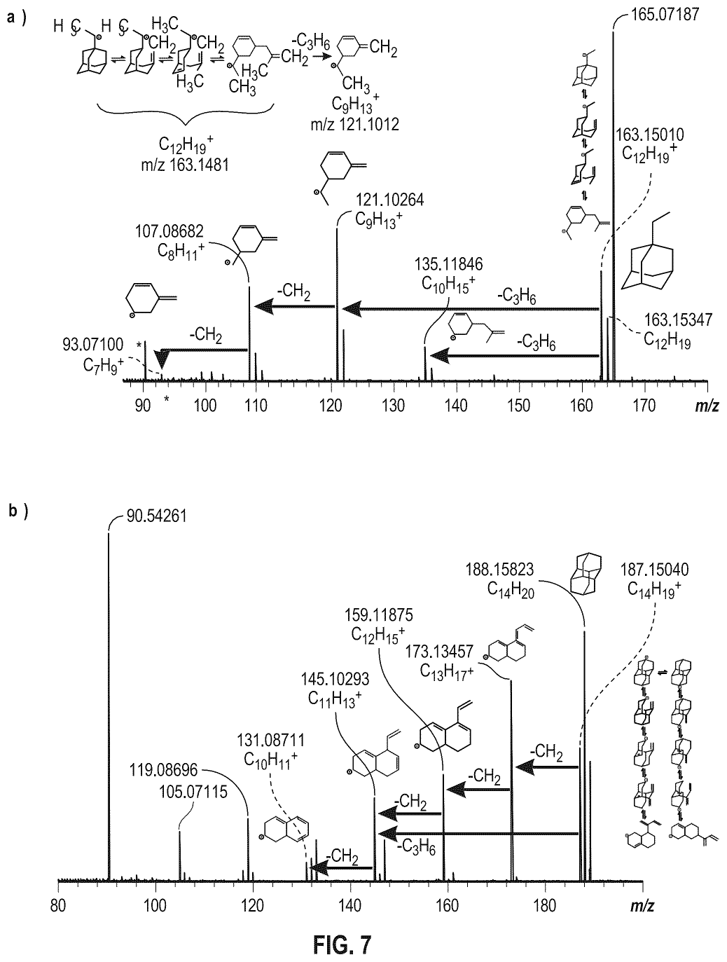

Experimentally, there is no report in the literature that the APPI source can ionize these structures (Oldenburg et al, 2014). For this purpose, fragmentation experiments of these structures were carried out in order to ensure that these structures ionize using APPI (+) FT-ICR MS. For this, the APCI source was used, as it already resembles EI ionization, which has already been widely discussed in the literature de fragment ions of these types of compounds; the ionization involves the mechanism of chemical ionization through the use of corona discharge. For this evaluation, the COP 96 sample was chosen because it presents a high thermal evolution, and an energy of 8 eV was used, with an isolation window of 2 Da. In FIG. 7, there can be observed the fragmentation a) of adamantane (m/z 136); b) ethyladamantane (164 m/z); c) diamantane (188 m/z), d) triamantane (139 m/z).

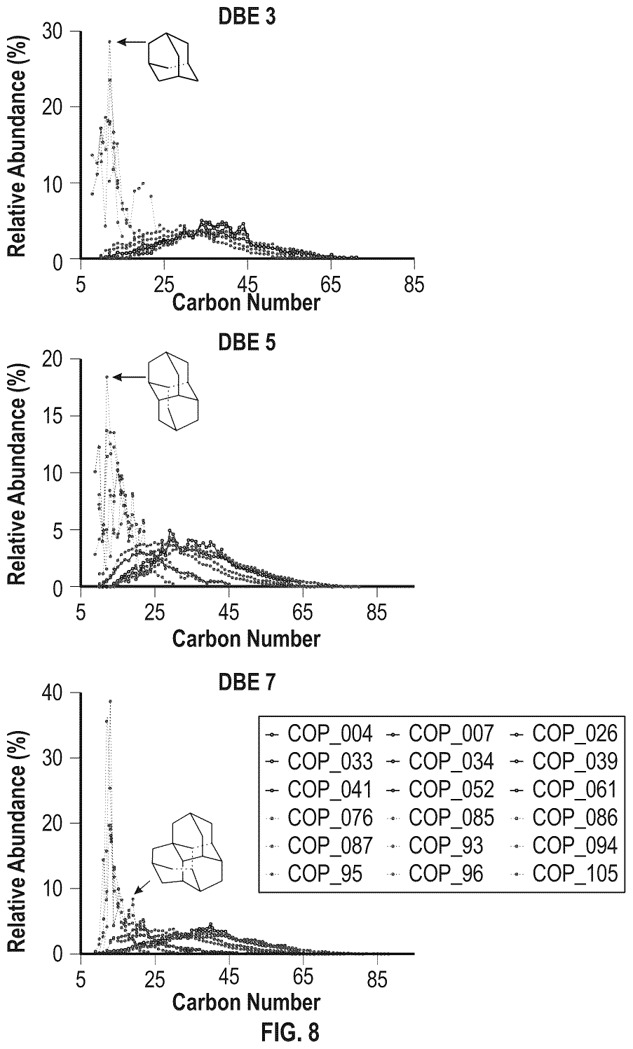

FIG. 8 illustrates the distribution of carbon atoms for the hydrocarbon species of DBE 3, 5 and 7. For DBE 3, an emergence of carbon atoms from 10 to 12 is observed, probably attributed to adamantane (C10H16), methyladamantane (C11H18) and ethyladamantane (C12H20), the latter being the most intense. For DBE 5, an emergence of carbon atoms 14 and 15 is observed, probably attributed to diamantane (C14H20) and methyldiamantane (C15H22). For DBE 7, an intermediate emergence is observed and can be attributed to triamantane (C18H24); in this DBE, there also can be other nuclei belonging to naphthalene (C10H8) and the monoaromatic steroids C17 and C27.

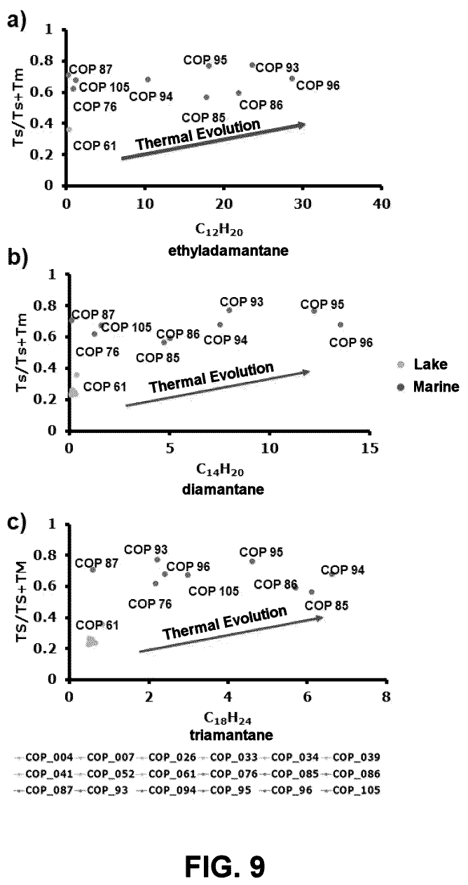

In FIG. 9 it is possible to compare the ratio of Ts/Ts+Tm for ethylamantane (a), diamantane (b) and triamantane (c). Ts/Ts+Tm is a parameter that applies to oils typically up to Ro(0.85) and diamondoids beyond that. From these figures, there can be observed that there is an increase in the relative abundance of this series of diamondoids in the more evolved samples, and in ethyladamantane (DBE 3) and diamantane (DBE 5) a greater similarity in the distribution profile of the samples is noticed; whereas triamantane, perhaps due to signal suppression of naphthalene ions that are very close to its ions, does not present a thermal evolution trend similar to other diamondoids.

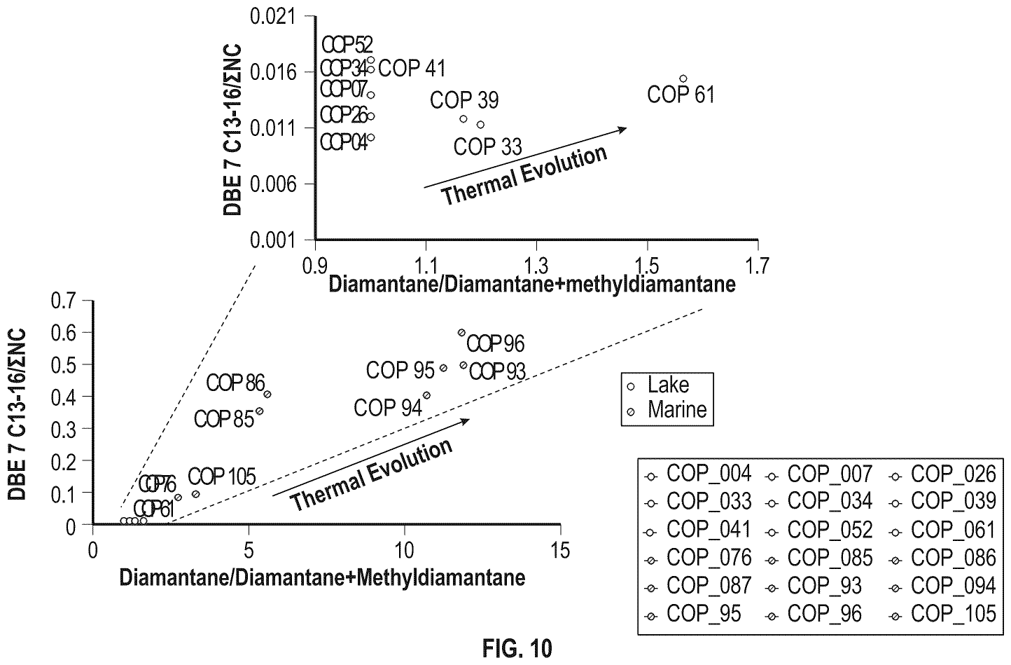

FIG. 10 shows a thermal evolution trend very consistent with those previously evaluated using the ratio of the sum of carbon atoms C13 to C16, probably associated with naphthalene; DBE 7, divided by the sum of the total carbon number by the ratio of diamantane to the sum of diamantane and methyldiamantane (C14/C14+C15; DBE 5). As previously, there can be seen the COP 96 sample being the most evolved of the samples. With the expansion to samples of lake origin, it is clear that sample COP 61 moves more intensely due to the diamond content, perhaps because it is a sample with a possible association with biodegradation, whereas the COP 52 sample presents the highest intensity for the ratio of the naphthalene alkyl series.

Validation of Evaluations with Hydropyrolysis Samples

Hydropyrolysis experiments are normally carried out in order to simulate the thermal evolution of source rocks, evaluate the extent of oil and bitumen formation and with the aim of calibrating the thermal history of the sedimentation basin by simulating physical conditions such as temperature and pressure (Mackenzie, et al., 1981; Seifert and Moldowan, 1978, 1980). In the experiment carried out at CENPES Petrobras with Type II-S kerogen samples, aliquots of samples were taken at temperature intervals, starting at 300° C. and ending at 365° C., at which the peak of the oil window was reached. Using ultra-high resolution mass spectrometry, with the APPI (+) ionization source, changes in the compositions of biomarkers and molecular markers of aromatic hydrocarbons were evaluated with the increase in the thermal evolution of hydropyrolysis samples with the aim of validating the main reasons used for samples from the Santos Basin.

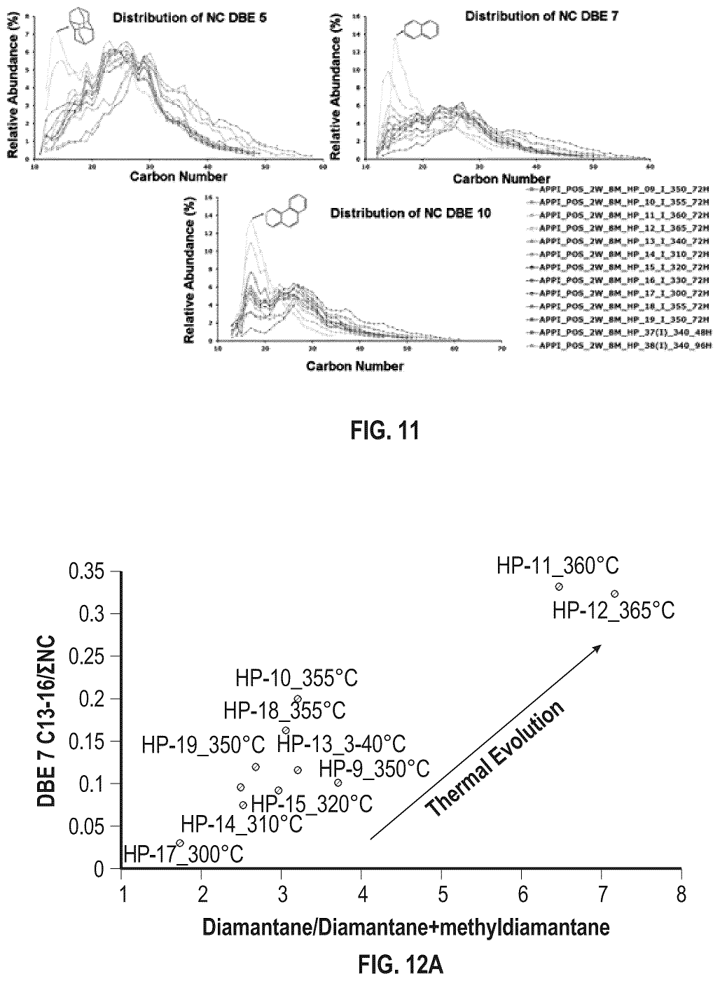

FIG. 11 illustrates the distributions of carbon atoms for the hydrocarbon species of DBEs 5, 7 and 10. For DBE 5, an emergence of carbon atoms 14 and 15 is observed, probably attributed to the diamantane (C14H20) and methyldiamantane (C15H22). For DBE 7, an increase in relative abundances is noted for the carbon atoms from 13 to 16 probably attributed to the naphthalene nucleus (C10H8), which may be linked to the ring as propyl, butyl and pentyl groups. In DBE 10, an increase in the abundance of carbon atoms from 14 to 16 is also shown, probably attributed to the phenanthrene nucleus (C14H10). All of these structures were previously used as markers of thermal evolution for samples from the Santos Basin and were found to be more abundant in more thermally evolved samples.

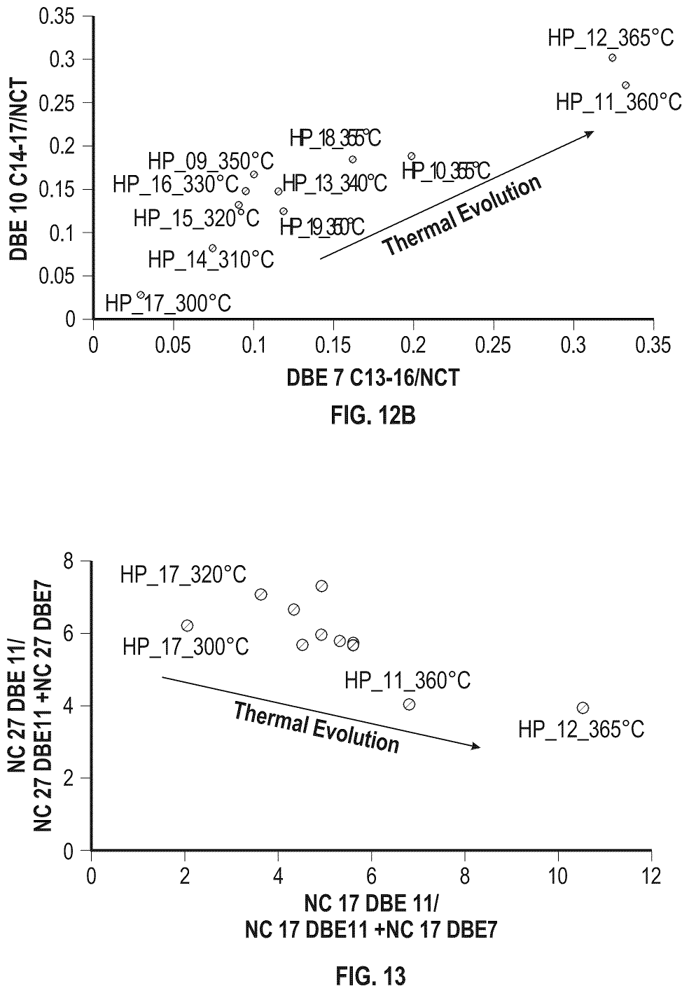

In FIG. 12a, the ratio of diadamantane (C14H20) to the sum of diamantane (C14H20) with methyldiadamantane (C15H22) was used, with the abundance normalized for DBE 5 in relation to naphthalene (C10H8) with the sum of the atoms of carbon from C13 to C16 by the sum of the normalized abundance of the number of total carbons of DBE 7. It is noted that, for samples at the extremes of the hydropyrolysis temperature 300° C., 360 and 365° C., this relation using these markers works very well. However, for some samples, there is no linear increase, leaving the parameters slightly distorted, especially for the HP_38_340° C. sample. For both markers, temperatures from 300 to 365° C. are discriminating, showing the potential for using these ratios; however, samples at temperatures from 320 to 350° C. showed little variation.

In FIG. 12b, greater linearity is observed with increasing temperature between the ratios of naphthalene (C10H8), DBE 7, with the sum of carbon atoms from C13 to C16 by the sum of the normalized abundance of the number of carbons totals of DBE 7 in relation to phenanthrene (C14H10), DBE 10, with the sum of carbon atoms from C14 to C17 by the normalized abundance of the total carbon number of DBE 10.

In FIG. 13, it is noted that for the ratio using steroids (TA/TA+TM), considering the carbon atoms C17 and 27, as already demonstrated in the Santos Basin, there is presented a greater correlation for the hydropyrolysis samples for the carbon atom C17. For C17, the more evolved samples had a higher ratio for DBE11, showing greater aromatization, whereas C27 did not show an aromatization trend, and there was even a decrease for the more evolved samples for this set of samples.

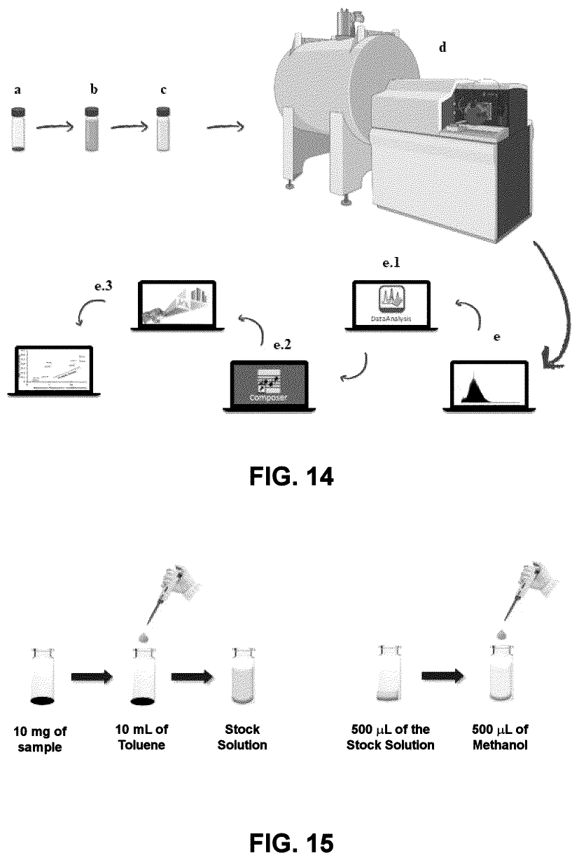

FIG. 14 depicts the components of the invention. In general terms, the method consists of the following steps: weighing the sample (a); dilution in toluene (b); addition of methanol (c); analysis of the oil solution by APPI(+) FT-ICR MS (d); spectrum processing (e); which consists of the recalibration of the raw spectrum using the DataAnalysis software (e.1); assignment of molecular formulas by the Composer software (e.2); and data analysis by the Thanus Software (e.3).

### Example

The invention proposed here consists of a method for obtaining two new proxies for evaluating thermal evolution. As seen in FIG. 15, in general terms, crude oil samples were prepared by weighing 10 mg of the oil and dissolving it in 10 mL of toluene. For APPI analyses, the final oil concentration is 500 mg·mL⁻¹ in toluene/methanol (50:50). The solvents methanol, toluene and ammonium hydroxide are HPLC grade acquired from J. T. Baker (Phillipsburg, NJ, USA).

Mass spectrometry analyses were carried out using an FT-ICR MS 7T SolariX 2xR equipment (Bruker Daltonics—Bremen, Germany) coupled to the ESI and APPI source. The equipment was calibrated daily with a solution of 0.1 mg·mL⁻¹ of NaTFA calibrant, for positive and negative mode, in the m/z range of 150 to 2000. The average calibration error varied between 0.02 and 0.04 ppm in linear regression mode. 8MW data sets files were acquired via magnitude mode with the detection range of m/z 150-2000. Typically, for each sample, a total of 300 scans were acquired to obtain spectra with excellent signal/noise values. The sodium trifluoroacetate (NaTFA) calibrant used to calibrate the mass spectrometer is from Sigma-Aldrich (Steinheim, Germany).

TABLE 1
Parameters used in the APPI(+) ionization
sources for sample acquisition.

| Source parameters | APPI(+) |
|---|---|
| Flow rate (μL · h⁻¹) | 500 |
| Capillary tension (kV) | 4.0 |
| Final Plate Displacement (V) | −500 |
| Source Gas Nebulizer (bar-x100 kPa) | 2.0 (200 kPa) |
| Ion source gas temperature (° C.) | 400 |
| Drying gas flow rate (L · min⁻¹) | 4.0 |
| Drying Gas Temperature (° C.) | 200 |
| Capillary Output (V) | 200 |
| Baffle Plate (V) | 220 |
| Funnel 1 | 150 |
| Skimmer (V) | 45 |
| Funnel RF Amplitude (Vpp) | 140 |
| Ion Accumulation Time (sec) | 0.010 |
| Collision cell | |
| Collision RF Amplitude (Vpp) | 1600 |
| Optical transfer | |
| Flight time (ms) | 1,200 |
| Frequency (MHz) | 4 |

Routinely, in petroleomics, data processing consists of three steps, as illustrated in FIG. 14, steps e.1 to e.3. Step e.1 refers to the internal recalibration of the raw spectrum. To do this, one of the hundreds of homologous series of the known constituents of oil is used. Step e.2 is the assignment of molecular formulas to the detected signals. This step is carried out with the help of software, such as Composer, PetroMS and PetroOrg. Step e.3 refers to the categorization of FT-ICR MS data using various graphical data visualization and interpretation tools with the help of the Thanus software, developed via a Cooperation Agreement established between UFG and Petrobras.

In step e.1—recalibration—the raw spectra obtained by the FT-ICR MS, 7T SolariX 2xR, were recalibrated internally using the DataAnalysis 5.0 SRI software (Version 5.0 Build 203.2.3586 64-bit Copyright© 2017 Bruker Daltonik GmbH).

Step e.2 of data processing consists of assigning molecular formulas based on the recalibrated spectra. To do this, the Composer 64 software (Version 1.5.3 Sierra Analytica, Modesto, USA) is used.

TABLE 2
Parameters used in processing the mass spectra of oil
samples using the Composer software for APPI(+).

| APPI(+) | |
|---|---|
| Recalibration method | |
| Equation | Path |
| | Recalibration |
| Tolerance window (ppm) | 1.00 |
| Intensity threshold (%) | 0.60 |
| Minimum m/z (Da) | 200 |
| Maximum m/z (Da) | 2000 |
| Closest match to theory | Yes |
| Homologous series | Automated |

Composition

| Allow radicals and adduction/ | Yes | |
|---|---|---|
| loss ions | | |
| DBE range | 0.0-40.0 | |
| Range m/z | 200-2000 | |
| Minimum abundance (%) | 0.60 | |
| Compute mode | Use of hydro- | |
| | carbon rules | |
| Upper limit m/z again | 500 | |
| Minimum abundance again (%) | 0.60 | |
| Element Ranges | C | 0-200 |
| | H | 0-1000 |
| | N | 0-3 |
| | O | 0-3 |
| | S | 0-3 |

In general, the processing conditions established were similar for all samples. However, the intensity threshold, minimum abundance and minimum abundance parameters varied according to the noise intensity of each spectrum and the used ionization source. These three parameters are used to define a relative abundance limit, so that molecular formulas were only assigned to peaks with an intensity higher than the pre-established limit, that is, 3 times higher than the spectrum noise. In this way, mistaken assignments for low-intensity signals, which could be noise, are avoided.

The composition data obtained in Composer are saved in csv format (separated by commas), which are used as input data in the Thanus software. The visualization can be related not only to the elaboration of different types of graphs but also to the simultaneous visualization of data from different samples, facilitating the interpretation of data and comparison of a set of samples.

Those skilled in the art in the technical field of organic geochemistry will value the knowledge presented herein and will be able to reproduce the invention in the presented embodiments and in other variants, encompassed by the scope of the attached claims.

1-8. (canceled)

9. A method for detecting aromatic hydrocarbons or diamondoids, the method comprising:
preparing an oil sample for analysis;
establishing one or more parameters for sample analysis;
conducting analysis of the prepared oil sample;
generating spectra corresponding to the oil sample;
recalibrating the spectrum data using at least one homologous series of known oil constituents;
assigning molecular formulae to the recalibrated raw spectrum data; and
identifying aromatic hydrocarbons or diamondoids present in the oil sample based on the assigned molecular formulae.

10. The method of claim 9, wherein preparing an oil sample comprises:
diluting oil in toluene; and
adding methanol to the oil diluted with toluene to an oil concentration of 500 mg/ml.

11. The method of claim 9, wherein conducting analysis of the prepared oil sample comprises using Fourier transform ion cyclotronic resonance mass spectrometry coupled with an atmospheric pressure photoionization source (APPI(+)-FT-ICR MS) to analyze the prepared oil sample.

12. The method of claim 9, wherein the oil sample is a crude oil sample.

13. The method of claim 11, wherein conducting analysis of the prepared oil sample using APPI(+)-FT-ICR MS is accomplished with a resolving power of about 800,000.

14. The method of claim 11, wherein establishing one or more parameters comprises setting the following:
Capillary tension: 4.0 kV;
Final plate displacement: −500 V;
Source gas nebulizer: 2.0 bar;
Ion source gas temperature: 400° C.;
Capillary Output: 200 V;
Baffle plate: 220 V;
Skimmer: 45 V;
Funnel RF amplitude: 140 Vpp;
Ion accumulation time: 0.010 sec;
Collision RF amplitude: 1600 Vpp;
Flight time: 1,200 ms; and
Frequency: 4 MHz.

15. The method of claim 9, wherein assigning molecular formulae to the recalibrated raw spectrum data comprises using the following parameters:
Tolerance window: 1.00 ppm;
Intensity Threshold: 0.60%;
Minimum m/z: 200 Da;
Maximum m/z: 2000 Da;
Minimum abundance: 0.60%; and
DBE range: DBE: 0.0-40.0.

16. The method of claim 16, wherein assigning molecular formulae to the recalibrated raw spectrum data is accomplished using the following element ranges:
Carbon: 0-200;
Hydrogen: 0-1000;
Nitrogen: 0-3;
Oxygen: 0-3; and
Sulfur: 0-3.

17. The method of claim 9, wherein assigning molecular formulae to the recalibrated raw spectrum data comprises assigning molecular formulae to signals in the spectrum data that have a peak intensity that is at least 3 times higher than a spectrum noise.
